(19) United States
(12) Patent Application Publication
(10) Pub. No.: US 2022/0047567 A1
(43) Pub. Date: Feb. 17, 2022

(54) METHODS FOR THE TREATMENT OF NEUROFIBROMATOSIS

(71) Applicants: INSERM (INSTITUT NATIONAL DE LA SANTÉ ET DE LA RECHERCHE MÉDICALE), Paris (FR); CENTRE NATIONAL DE LA RECHERCHE SCIENTIFIQUE (CNRS), Paris (FR); ASSISTANCE PUBLIQUE-HÔPITAUX DE PARIS (APHP), Paris (FR); UNIVERSITÉ DE PARIS, Paris (FR)

(72) Inventor: Guillaume CANAUD, Paris (FR)

(21) Appl. No.: 17/274,600

(22) PCT Filed: Sep. 9, 2019
(86) PCT No.: PCT/EP2019/073945

§ 371 Date: Mar. 9, 2021

(30) Foreign Application Priority Data
Sep. 10, 2018 (EP) 18306186.0

(51) Int. Cl. A61K 31/4439; A61K 45/06

(52) U.S. Cl. CPC A61K 31/4439; A61K 45/06

(57) ABSTRACT

The invention relates to a PI3K inhibitor for use in the treatment of neurofibromatosis type 1 and type 2 in a subject in need thereof. Currently there are no treatment of neurofibromatosis type 1 and 2. Patients mainly received supportive care to treat severe symptoms including surgery to remove tumors compressing nearby tissue or damaging organs, stereotactic radiosurgery or cochlear implants. Inventors have worked on immortalized NF1−/− cells and shown that PIK3CA pharmacological inhibition (BYL719) was associated with increased apoptosis as assessed by PARP cleavage in a dose dependent manner. They also decided to expose NF1−/− cells to a multitargeted therapy including BYL719+selumetinib or BYL719+selumetinib+IPA-3. Importantly, they found that both combinations led to severe apoptosis with double strand DNA break as assessed by the phosphorylation of H2aX. This new approach with either BYL719 alone or in combination with other therapeutics seem to be very promising in patients with neurofibromatosis.

### FIELD OF THE INVENTION

The invention relates to methods and compositions for the treatment of neurofibromatosis.

### BACKGROUND OF THE INVENTION

Neurofibromatosis type 1 (NF1) and type 2 (NF2) are genetically distinct disorders that cause a multitude of disease manifestations. Affecting both children and adults, these 2 diseases and other phenotypically similar conditions often result in devastating complications that are in great part untreatable. Most adults with neurofibromatosis type 1 develop neurofibromas, which are noncancerous (benign) tumors that are usually located on or just under the skin (Kresak J L et al, J Pediatr Genet 2016). These tumors may also occur in nerves near the spinal cord or along nerves elsewhere in the body. Some people with neurofibromatosis type 1 may develop cancerous tumors that grow along nerves. These tumors, which usually develop in adolescence or adulthood, are called malignant peripheral nerve sheath tumors. People with neurofibromatosis type 1 also have an increased risk of developing other cancers, including brain tumors and leukemia (Kresak J L et al, J Pediatr Genet 2016). During childhood, benign growths called Lisch nodules often appear in the iris. Lisch nodules do not interfere with vision. Some affected individuals also develop tumors that grow along the optic nerve. These tumors, which are called optic gliomas, may lead to reduced vision or total vision loss. In some cases, optic gliomas have no effect on vision. Additional signs and symptoms of neurofibromatosis type 1 include hypertension, short stature, macrocephaly, and skeletal abnormalities such as an abnormal curvature of the spine. Neurofibromatosis type 1 occurs in 1 in 3,000 to 4,000 people worldwide (Kresak J L et al, J Pediatr Genet 2016).

The NF1 gene provides instructions for making a protein called neurofibromin. This protein is produced in many cells, including nerve cells and specialized cells surrounding nerves (oligodendrocytes and Schwann cells). Neurofibromin acts as a tumor suppressor, which means that it keeps cells from growing and dividing too rapidly or in an uncontrolled way. Mutations in the NF1 gene lead to the production of a nonfunctional version of neurofibromin that cannot regulate cell growth and division. As a result, tumors such as neurofibromas can form along nerves throughout the body. It is unclear how mutations in the NF1 gene lead to the other features of neurofibromatosis type 1, such as café-au-lait spots and learning disabilities.

Neurofibromatosis type 1 is considered to have an autosomal dominant pattern of inheritance. People with this condition are born with one mutated copy of the NF1 gene in each cell. In about half of cases, the altered gene is inherited from an affected parent. The remaining cases result from new mutations in the NF1 gene and occur in people with no history of the disorder in their family. Importantly, two copies of the NF1 gene must be altered to trigger tumor formation in neurofibromatosis type 1. A mutation in the second copy of the NF1 gene occurs during a person's lifetime in specialized cells surrounding nerves. Almost everyone who is born with one NF1 mutation acquires a second mutation in many cells and develops the tumors characteristic of neurofibromatosis type 1.

The signs and symptoms of neurofibromatosis type 2 usually appear during adolescence or in a person's early twenties, although they can begin at any age (Kresak J L et al, J Pediatr Genet 2016). The most frequent early symptoms of vestibular schwannomas are hearing loss, ringing in the ears (tinnitus), and problems with balance. In most cases, these tumors occur in both ears by age 30. If tumors develop elsewhere in the nervous system, signs and symptoms vary according to their location. Complications of tumor growth can include changes in vision, numbness or weakness in the arms or legs, and fluid buildup in the brain. Some people with neurofibromatosis type 2 also develop clouding of the lens (cataracts) in one or both eyes, often beginning in childhood. Neurofibromatosis type 2 has an estimated incidence of 1 in 33,000 people worldwide. Mutations in the NF2 gene cause neurofibromatosis type 2. The NF2 gene provides instructions for making a protein called merlin (also known as schwannomin). This protein is produced in the nervous system, particularly in Schwann cells, which surround and insulate nerve cells (neurons) in the brain and spinal cord. Merlin acts as a tumor suppressor, which means that it keeps cells from growing and dividing too rapidly or in an uncontrolled way. Although its exact function is unknown, this protein is likely also involved in controlling cell movement, cell shape, and communication between cells. Mutations in the NF2 gene lead to the production of a nonfunctional version of the merlin protein that cannot regulate the growth and division of cells. Research suggests that the loss of merlin allows cells, especially Schwann cells, to multiply too frequently and form the tumors characteristic of neurofibromatosis type 2.

Neurofibromatosis type 2 is considered to have an autosomal dominant pattern of inheritance. People with this condition are born with one mutated copy of the NF2 gene in each cell. In about half of cases, the altered gene is inherited from an affected parent. The remaining cases result from new mutations in the NF2 gene and occur in people with no history of the disorder in their family. Such as for NF1, a second hit is necessary to alter the second copy of the gene and to lead to tumor formation.

Currently there are no treatment of neurofibromatosis type 1 and 2. Patients mainly received supportive care to treat severe symptoms including surgery to remove tumors compressing nearby tissue or damaging organs, stereotactic radiosurgery or cochlear implants. Malignant tumors and other cancers associated with neurofibromatosis are treated with standard cancer therapies. Accordingly, there is a need to find new strategies to treat neurofibromatosis type 1 and 2.

### SUMMARY OF THE INVENTION

The present invention relates to a PI3K inhibitor for use in the treatment of neurofibromatosis type 1 and type 2 in a subject in need thereof. In particular, the present invention is defined by the claims.

### DETAILED DESCRIPTION OF THE INVENTION

Inventors have worked on immortalized NF1−/− cells and shown that PIK3CA pharmacological inhibition (e.g. BYL719) was associated with increased apoptosis as assessed by PARP cleavage in a dose dependent manner. They also exposed the NF1−/− cells to different dose of selumetinib, a MAP kinase inhibitor. Interestingly they observed that selumetinib induced apoptosis in a dose dependent manner but to a lower extent compared to BYL719. Based on these results, they decided to expose NF1−/− cells to a multi-targeted therapy including BYL719+selumetinib or BYL719+selumetinib+IPA-3 (PAK inhibitor). Importantly, they found that both combinations led to severe apoptosis with double strand DNA break as assessed by the phosphorylation of H2aX.

This new approach with either BYL719 alone or in combination with other therapeutics seem to be very promising in patients with neurofibromatosis.

Accordingly, in a first aspect, the present invention relates to a PI3K inhibitor for use in the treatment of neurofibromatosis type 1 and type 2 in a subject in need thereof.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein the term “neurofibromatosis” refers to a rare genetic disorder that causes typically benign tumors on or just under the skin and also in nerves near the spinal cord or along nerves elsewhere in the body. There are three types of Neurofibromatosis (NF): neurofibromatosis type 1 (NF1), neurofibromatosis type 2 (NF2), and schwannomatosis. The neurofibromatosis type 1 (NF1) is caused by mutations in the NF1 gene lead to the production of a nonfunctional version of neurofibromin that cannot regulate cell growth and division. As a result, tumors such as neurofibromas can form along nerves throughout the body. NF-1 is an autosomal dominant disorder. The neurofibromatosis type 2 (NF2) is caused by mutations in the NF2 gene cause neurofibromatosis type 2. NF2 also known as MISME syndrome for multiple inherited schwannomas, meningiomas, and ependymomas. Signs and symptoms of NF2 usually result from the development of benign, slow-growing tumors (acoustic neuromas) in both ears. Also known as vestibular schwannomas, these tumors grow on the nerve that carries sound and balance information from the inner ear to the brain. Schwannomatosis causes tumors to develop on skull (cranial), spinal and peripheral nerves but not on the nerve that carries sound and balance information from the inner ear to the brain.

As used herein, the term “subject” refers to any mammals, such as a rodent, a feline, a canine, and a primate. Particularly, in the present invention, the subject is a human afflicted with or susceptible to be afflicted with neurofibromatosis type 1. In another embodiment, the subject is a human afflicted with or susceptible to be afflicted with neurofibromatosis type 2 as described above.

As used herein, the term “PI3K refers to phosphoinositide 3-kinases also called phophatidylinositide 3-kinases. PI3K belongs to a family of enzymes which phosphorylate the 3′hydroxyl group of the onositol ring of the phosphatidylinositol (PtdIns). The PI3K signalling pathway can be activated, resulting in the synthesis of PIP3 from PIP2. The PI3K family is divided into four different classes: Class I, Class II, Class III, and Class IV. PI3K is involved in the control multiple cellular processes including metabolism, motility, proliferation, growth, and survival, is one of the most frequently dysregulated pathways in human cancers.

As used herein, the term “PI3K inhibitor” refers to a natural or synthetic compound that has a biological effect to inhibit the activity or the expression of PI3K. More particularly, such compound is capable of inhibiting the kinase activity of at least one member of PI3K family, for example, at least a member of Class I PI3K. In particular embodiment, said PI3K inhibitor may be a pan-inhibitor of Class I PI3K (known as p110) or isoform specific of Class I PI3K isoforms (among the four types of isoforms, p110α, p110β, p110γ or p110δ).

In a particular embodiment, the PI3K inhibitor is a peptide, petptidomimetic, small organic molecule, antibody, aptamers, siRNA or antisense oligonucleotide. The term “peptidomimetic” refers to a small protein-like chain designed to mimic a peptide. In a particular embodiment, the inhibitor of PI3K is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity.

In a particular embodiment, the PI3K inhibitor is a small organic molecule. The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

In a particular embodiment, the PI3K inhibitor is a small molecule which is an isoform-selective inhibitor of PI3K selected among the following compounds: BYL719 (Alpelisib, Novartis), GDC-0032 (Taselisib, Genentech/Roche), BKM120 (Buparlisib), INK1117 (Millenium), A66 (University of Auckland), GSK260301 (Glaxosmithkline), KIN-193 (Astra-Zeneca), TGX221 (Monash University), TG1202, CAL101 (Idelalisib, Gilead Sciences), GS-9820 (Gilead Sciences), AMG319 (Amgen), IC87114 (Icos Corporation), BAY80-6946 (Copanlisib, Bayer Healthcare), GDC0941 (Pictlisib, Genentech), IPI145 (Duvelisib, Infinity), SAR405 (Sanofi), PX-866 (Oncothyreon) or their pharmaceutically acceptable salts. Such PI3K inhibitors are well-known in the art and described for example in Wang et al Acta Pharmacological Sinica (2015) 36: 1170-1176.

In a particular embodiment, the PI3K inhibitor is BYL719. As used herein, the term “BYL719” is an ATP-competitive oral PI3K inhibitor selective for the p110a isoform that is activated by a mutant PIK3CA gene (Furet P., et al. 2013; Fritsch C., et al 2014). This molecule is also called Alpelisib and has the following formula and structure in the art C₁₉H₂₂F₃N₅O₂S:

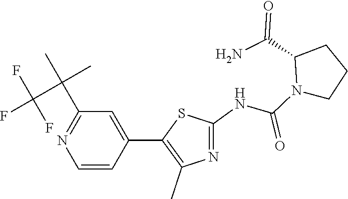 [SMILES: Cc1nc(NC(=O)N2CCC[C@H]2C(N)=O)sc1-c1ccnc(C(C)(C)C(F)(F)F)c1]

In a particular embodiment, the PI3K inhibitor is GDC-0032, developed by Roche. This molecule also called Taselisib has the following formula and structure in the art C₂₄H₂₈N₈O₂:

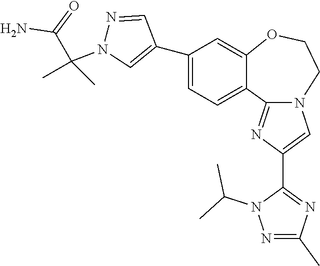 [SMILES: Cc1nc(-c2cn3c(n2)-c2ccc(-c4cnn(C(C)(C)C(N)=O)c4)cc2OCC3)n(C(C)C)n1]

In some embodiments, the PI3K inhibitor is an antibody. As used herein, the term “antibody” is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity. The term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody is a “chimeric” antibody as described in U.S. Pat. No. 4,816,567. In some embodiments, the antibody is a humanized antibody, such as described U.S. Pat. Nos. 6,982,321 and 7,087,409. In some embodiments, the antibody is a human antibody. A “human antibody” such as described in U.S. Pat. Nos. 6,075,181 and 6,150,584. In some embodiments, the antibody is a single domain antibody such as described in EP 0 368 684, WO 06/030220 and WO 06/003388. In a particular embodiment, the inhibitor is a monoclonal antibody. Monoclonal antibodies can be prepared and isolated using any technique that provides for the production of antibody molecules by continuous cell lines in culture. Techniques for production and isolation include but are not limited to the hybridoma technique, the human B-cell hybridoma technique and the EBV-hybridoma technique.

In a particular, the PI3K inhibitor is an intrabody having specificity for PI3K. As used herein, the term “intrabody” generally refer to an intracellular antibody or antibody fragment. Antibodies, in particular single chain variable antibody fragments (scFv), can be modified for intracellular localization. Such modification may entail for example, the fusion to a stable intracellular protein, such as, e.g., maltose binding protein, or the addition of intracellular trafficking/localization peptide sequences, such as, e.g., the endoplasmic reticulum retention. In some embodiments, the intrabody is a single domain antibody. In some embodiments, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

In some embodiments, the PI3K inhibitor is a short hairpin RNA (shRNA), a small interfering RNA (siRNA) or an antisense oligonucleotide which inhibits the expression of USP14. In a particular embodiment, the inhibitor of JMY expression is siRNA. A short hairpin RNA (shRNA) is a sequence of RNA that makes a tight hairpin turn that can be used to silence gene expression via RNA interference. shRNA is generally expressed using a vector introduced into cells, wherein the vector utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA to which it is bound. Small interfering RNA (siRNA), sometimes known as short interfering RNA or silencing RNA, are a class of 20-25 nucleotide-long double-stranded RNA molecules that play a variety of roles in biology. Most notably, siRNA is involved in the RNA interference (RNAi) pathway whereby the siRNA interferes with the expression of a specific gene. Anti-sense oligonucleotides include anti-sense RNA molecules and anti-sense DNA molecules, would act to directly block the translation of the targeted mRNA by binding thereto and thus preventing protein translation or increasing mRNA degradation, thus decreasing the level of the targeted protein, and thus activity, in a cell. For example, antisense oligonucleotides of at least about 15 bases and complementary to unique regions of the mRNA transcript sequence can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Antisense oligonucleotides, siRNAs, shRNAs of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically mast cells. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

In some embodiments, the inhibitor of PI3K expression is an endonuclease. In the last few years, staggering advances in sequencing technologies have provided an unprecedentedly detailed overview of the multiple genetic aberrations in cancer. By considerably expanding the list of new potential oncogenes and tumor suppressor genes, these new data strongly emphasize the need of fast and reliable strategies to characterize the normal and pathological function of these genes and assess their role, in particular as driving factors during oncogenesis. As an alternative to more conventional approaches, such as cDNA overexpression or downregulation by RNA interference, the new technologies provide the means to recreate the actual mutations observed in cancer through direct manipulation of the genome. Indeed, natural and engineered nuclease enzymes have attracted considerable attention in the recent years. The mechanism behind endonuclease-based genome inactivating generally requires a first step of DNA single or double strand break, which can then trigger two distinct cellular mechanisms for DNA repair, which can be exploited for DNA inactivating: the errorprone nonhomologous end joining (NHEJ) and the high-fidelity homology-directed repair (HDR).

In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences.

In some embodiment, the endonuclease is CRISPR-cas9 which is from Streptococcus pyogenes. The CRISPR/Cas9 system has been described in U.S. Pat. No. 8,697,359 B1 and US 2014/0068797. Originally an adaptive immune system in prokaryotes (Barrangou and Marraffini, 2014), CRISPR has been recently engineered into a new powerful tool for genome editing. It has already been successfully used to target important genes in many cell lines and organisms, including human (Mali et al., 2013, Science, Vol. 339: 823-826), bacteria (Fabre et al., 2014, PLoS Negl. Trop. Dis., Vol. 8:e2671.), zebrafish (Hwang et al., 2013, PLoS One, Vol. 8:e68708.), C. elegans (Hai et al., 2014 Cell Res. doi: 10.1038/cr.2014.11.), bacteria (Fabre et al., 2014, PLoS Negl. Trop. Dis., Vol. 8:e2671.), plants (Mali et al., 2013, Science, Vol. 339: 823-826), Xenopus tropicalis (Guo et al., 2014, Development, Vol. 141: 707-714.), yeast (DiCarlo et al., 2013, Nucleic Acids Res., Vol. 41: 4336-4343.), Drosophila (Gratz et al., 2014 Genetics, doi:10.1534/genetics.113.160713), monkeys (Niu et al., 2014, Cell, Vol. 156: 836-843.), rabbits (Yang et al., 2014, J. Mol. Cell Biol., Vol. 6: 97-99.), pigs (Hai et al., 2014, Cell Res. doi: 10.1038/cr.2014.11.), rats (Ma et al., 2014, Cell Res., Vol. 24: 122-125.) and mice (Mashiko et al., 2014, Dev. Growth Differ. Vol. 56: 122-129.). Several groups have now taken advantage of this method to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. A recent exciting development is the use of the dCas9 version of the CRISPR/Cas9 system to target protein domains for transcriptional regulation, epigenetic modification, and microscopic visualization of specific genome loci.

In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

In a particular embodiment, the PI3K inhibitor for use according to the invention, and a MAPK, PAK, mTOR, TK, PARP or EGFR inhibitor as a combined preparation for simultaneous, separate or sequential use in the treatment of neurofibromatosis type 1 and type 2 in a subject in need thereof.

In another embodiment, the invention relates to a combination comprising a PI3K inhibitor, and at least one inhibitor selected from the group consisting of MAPK, PAK, mTOR, TK, PARP or EGFR inhibitors as described below for use in the treatment of neurofibromatosis type 1 and type 2 in a subject in need thereof.

In a particular embodiment, the PI3K inhibitor for use according to the invention, and MAPK inhibitor as a combined preparation for simultaneous, separate or sequential use in the treatment of neurofibromatosis type 1 and type 2 in a subject in need thereof.

As used herein, the term “MAPK” refers to mitogen-activated protein kinase, is a type of protein kinase that is specific to the amino acids serine and threonine. MAPK are involved in cellular responses to a diverse array of stimuli, such as mitogens, osmotic stress, heat shock and proinflammatory cytokines. Six groups of MAPK have so far been identified: Extracellular signal-regulated kinases (ERK1, ERK2), c-Jun N-terminal kinases (JNKs), p38 isoforms (MAPK11, MAPK12, MAPK13, MAPK14), ERK5 (MAPK7), ERK3 (MAPK6) and ERK4 (MAPK4), ERK7/8 (MAPK15). In a particular embodiment, the inhibitors of MAPK are inhibitors of ERK1/ERK2. The inhibitor of ERK1/ERK2 is selected from the group but is not limited to VTX-11e, SCH772984.

In a particular embodiment, the MAPK inhibitor is a peptide, petptidomimetic, small organic molecule, antibody, aptamers, siRNA or antisense oligonucleotide. In a particular embodiment, the MAPK inhibitor is p38-MAPK inhibitor. Typically, the inhibitor of p38-MAPK is selected from the group consisting of SB 203580, SB 203580 hydrochloride, SB681323 (Dilmapimod), LY2228820 dimesylate, BIRB 796 (Doramapimod), BMS-582949, Pamapimod, GW856553, ARRY-797AL 8697, AMG 548, CMPD-1, EO 1428, JX 401, RWJ 67657, TA 01, TA 02, VX 745, DBM 1285 dihydrochloride, ML 3403, SB 202190, SB 239063, SB 706504, SCIO 469 hydrochloride, SKF 86002 dihydrochloride, SX 011, TAK 715, VX 702, or PH-797804.

In a particular embodiment, the inhibitor of MAPK is an inhibitor of MEK. MEK1 and MEK2 are members of a larger family of dual-specificity kinases (MEK1-7) that phosphorylate threonine and tyrosine residues of various MAP kinases. In a particular embodiment, the inhibitor of MAPK is selected from the group consisting of Trametinib (GSK1120212); Selumetinib (AZD6244).

In a particular embodiment, the PI3K inhibitor for use according to the invention and, a MAPK inhibitor, as a combined preparation for simultaneous, separate or sequential use in the treatment of neurofibromatosis type 1 and type 2 in a subject in need thereof, wherein the PI3K inhibitor is BYL719 and, the MAPK inhibitor is Selumetinib.

In another embodiment, the PI3K inhibitor for use according to the invention, and PAK inhibitor as a combined preparation for simultaneous, separate or sequential use in the treatment of neurofibromatosis type 1 and type 2 in a subject in need thereof.

As used herein, the term “PAK” refers to p21-activated kinase which regulates cytoskeleton remodeling, phenotypic signaling and gene expression, and affects a wide variety of cellular processes such as directional motility, invasion, metastasis, growth, cell cycle progression, angiogenesis. In a particular embodiment, the PAK inhibitor is a peptide, petptidomimetic, small organic molecule, antibody, aptamers, siRNA or antisense oligonucleotide.

In a particular embodiment, the inhibitor of PAK is selected from the group consisting of PP1, hPIP1, NESH, Merlin, CRIPak, LKB1, Mesalamine, Glaucarubinone, Myricetin, β-elemene, miR-7, miR-let-7, miR-145, FRAX1036, OSU-03012, and IPA-3.

In a particular embodiment, the PAK inhibitor is used with thalidomide, lenalidomide or pomalidomide, as a combined preparation for use in the treatment of neurofibromatosis type 1 and type 2.

In a particular embodiment, the PI3K inhibitor for use according to the invention and, a PAK inhibitor, as a combined preparation for simultaneous, separate or sequential use in the treatment of neurofibromatosis type 1 and type 2 in a subject in need thereof, wherein the PI3K inhibitor is BYL719 and the PAK inhibitor is IPA-3.

In another embodiment, the PI3K inhibitor for use according to the invention, and mTOR inhibitor as a combined preparation for simultaneous, separate or sequential use in the treatment of neurofibromatosis type 1 and type 2 in a subject in need thereof.

As used herein, the term “mTOR” refers to mammalian target of rapamycin also known as mechanistic target of rapamycin and FK506-binding protein 12-rapamycin-associated protein 1 (FRAP1). mTOR functions as a serine/threonine protein kinase that regulates cell growth, cell proliferation, cell motility, cell survival, protein synthesis, autophagy, and transcription. mTOR has two structurally distinct complexes: mTORC1 and mTORC2. In a particular embodiment, the mTOR inhibitor is a peptide, petptidomimetic, small organic molecule, antibody, aptamers, siRNA or antisense oligonucleotide.

In a particular embodiment, the inhibitor of mTOR is selected from the group consisting of rapamycin (also called sirolimus and described in U.S. Pat. No. 3,929,992), temsirolimus, deforolimus, everolimus, tacrolimus and rapamycin analogue or derivative thereof, AMG954, AZD8055, AZD2014, BEZ235, BGT226, CC-115, CC-223, LY3023414, P7170, DS-7423, OSI-027, GSK2126458, PF-04691502, PF-05212384, INK128, MLN0128, MLN1117, Ridaforolimus, Metformin, XL765, SAR245409, SF1126, VS5584, GDC0980 and GSK2126458.

As used herein, the term “rapamycin analogue or derivative thereof” includes compounds having the rapamycin core structure as defined in U.S. Patent Application Publication No. 2003/0008923 (which is herein incorporated by reference), which may be chemically or biologically modified while still retaining mTOR inhibiting properties. Such derivatives include esters, ethers, oximes, hydrazones, and hydroxylamines of rapamycin, as well as compounds in which functional groups on the rapamycin core structure have been modified, for example, by reduction or oxidation. Pharmaceutically acceptable salts of such compounds are also considered to be rapamycin derivatives. Specific examples of esters and ethers of rapamycin are esters and ethers of the hydroxyl groups at the 42- and/or 31-positions of the rapamycin nucleus, and esters and ethers of a hydroxyl group at the 27-position (following chemical reduction of the 27-ketone). Specific examples of oximes, hydrazones, and hydroxylamines are of a ketone at the 42-position (following oxidation of the 42-hydroxyl group) and of 27-ketone of the rapamycin nucleus.

Examples of 42- and/or 31-esters and ethers of rapamycin are disclosed in the following patents, which are hereby incorporated by reference in their entireties: alkyl esters (U.S. Pat. No. 4,316,885); aminoalkyl esters (U.S. Pat. No. 4,650,803); fluorinated esters (U.S. Pat. No. 5,100,883); amide esters (U.S. Pat. No. 5,118,677); carbamate esters (U.S. Pat. No. 5,118,678); silyl ethers (U.S. Pat. No. 5,120,842); aminoesters (U.S. Pat. No. 5,130,307); acetals (U.S. Pat. No. 551,413); aminodiesters (U.S. Pat. No. 5,162,333); sulfonate and sulfate esters (U.S. Pat. No. 5,177,203); esters (U.S. Pat. No. 5,221,670); alkoxyesters (U.S. Pat. No. 5,233,036); O-aryl, -alkyl, -alkenyl, and -alkynyl ethers (U.S. Pat. No. 5,258,389); carbonate esters (U.S. Pat. No. 5,260,300); arylcarbonyl and alkoxycarbonyl carbamates (U.S. Pat. No. 5,262,423); carbamates (U.S. Pat. No. 5,302,584); hydroxyesters (U.S. Pat. No. 5,362,718); hindered esters (U.S. Pat. No. 5,385,908); heterocyclic esters (U.S. Pat. No. 5,385,909); gem-disubstituted esters (U.S. Pat. No. 5,385,910); amino alkanoic esters (U.S. Pat. No. 5,389,639); phosphorylcarbamate esters (U.S. Pat. No. 5,391,730); carbamate esters (U.S. Pat. No. 5,411,967); carbamate esters (U.S. Pat. No. 5,434,260); amidino carbamate esters (U.S. Pat. No. 5,463,048); carbamate esters (U.S. Pat. No. 5,480,988); carbamate esters (U.S. Pat. No. 5,480,989); carbamate esters (U.S. Pat. No. 5,489,680); hindered N-oxide esters (U.S. Pat. No. 5,491,231); biotin esters (U.S. Pat. No. 5,504,091); O-alkyl ethers (U.S. Pat. No. 5,665,772); and PEG esters of rapamycin (U.S. Pat. No. 5,780,462).

Examples of 27-esters and ethers of rapamycin are disclosed in U.S. Pat. No. 5,256,790, which is hereby incorporated by reference in its entirety.

Examples of oximes, hydrazones, and hydroxylamines of rapamycin are disclosed in U.S. Pat. Nos. 5,373,014, 5,378,836, 5,023,264, and 5,563,145, which are hereby incorporated by reference. The preparation of these oximes, hydrazones, and hydroxylamines is disclosed in the above listed patents. The preparation of 42-oxorapamycin is disclosed in U.S. Pat. No. 5,023,263, which is hereby incorporated by reference.

Other compounds within the scope of “rapamycin analog or derivative thereof” include those compounds and classes of compounds referred to as “rapalogs” in, for example, WO 98/02441 and references cited therein, and “epirapalogs” in, for example, WO 01/14387 and references cited therein.

Another compound within the scope of “rapamycin derivatives” is everolimus, a 4-O-(2-hydroxyethyl)-rapamycin derived from a macrolide antibiotic produced by Streptomyces hygroscopicus (Novartis). Everolimus is also known as Certican, RAD-001 and SDZ-RAD. Another preferred mTOR inhibitor is zotarolimus, an antiproliferative agent (Abbott Laboratories). Zotarolimus is believed to inhibit smooth muscle cell proliferation with a cytostatic effect resulting from the inhibition of mTOR. Another preferred mTOR inhibitor is tacrolimus, a macrolide lactone immunosuppressant isolated from the soil fungus Streptomyces tsukubaensis. Tacrolimus is also known as FK 506, FR 900506, Fujimycin, L 679934, Tsukubaenolide, PROTOPIC and PROGRAF. Other preferred mTOR inhibitors include AP-23675, AP-23573, and AP-23841 (Ariad Pharmaceuticals).

Preferred rapamycin derivatives include everolimus, CCI-779 (rapamycin 42-ester with 3-hydroxy-2-(hydroxymethyl)-2-methylpropionic acid; U.S. Pat. No. 5,362,718); 7-epi-rapamycin; 7-thiomethyl-rapamycin; 7-epi-trimethoxyphenyl-rapamycin; 7-epi-thiomethyl-rapamycin; 7-demethoxy-rapamycin; 32-demethoxy-rapamycin; 2-desmethyl-rapamycin; and 42-O-(2-hydroxy)ethyl rapamycin (U.S. Pat. No. 5,665,772).

Additional mTORC2 inhibitors may be OSI-027 (OSI Pharmaceuticals), a small molecule mTORC2 inhibitor. OSI-027 inhibits mTORC2 signaling complexes, allowing for the potential for complete truncation of aberrant cell signaling through this pathway.

In addition, torkinibs, ATP-competitive mTOR kinase domain inhibitors and inhibitors of mTORC2 may also be used according to the invention. Exemplary torkinibs include PP242 and PP30 (see, Feldman et al. (2009) PLoS Biology 7:371) and Torin1 (Thoreen et al. (2009) J Biol Chem 284:8023).

In a particular embodiment, the PI3K inhibitor for use according to the invention and, a mTOR inhibitor, as a combined preparation for simultaneous, separate or sequential use in the treatment of neurofibromatosis type 1 and type 2 in a subject in need thereof, wherein the PI3K inhibitor is BYL719 and the mTOR inhibitor is evorlimus.

In another embodiment, the PI3K inhibitor for use according to the invention, and tyrosine kinase inhibitor (TKI) as a combined preparation for simultaneous, separate or sequential use in the treatment of neurofibromatosis type 1 and type 2 in a subject in need thereof.

As used herein, the term “TKI” refers to tyrosine kinase inhibitor. Tyrosine kinase is involved in the phosphorylation of many proteins. Example of tyrosine kinase proteins: AATK; ABL; ABL2; ALK; AXL; BLK; BMX; BTK; CSF1R; CSK; DDR1; DDR2; EGFR; EPHA1; EPHA2; EPHA3; EPHA4; EPHA5; EPHA6; EPHA7; EPHA8; EPHA10; EPHB1; EPHB2; EPHB3; EPHB4; EPHB6; ERBB2; ERBB3; ERBB4; FER; FES; FGFR1; FGFR2; FGFR3; FGFR4; FGR; FLT1; FLT3; FLT4; FRK; FYN; GSG2; HCK; IGF1R; ILK; INSR; INSRR; IRAK4; ITK; JAK1; JAK2; JAK3; KDR; KIT; KSR1; LCK; LMTK2; LMTK3; LTK; LYN; MATK; MERTK; MET; MLTK; MST1R; MUSK; NPR1; NTRK1; NTRK2; NTRK3; PDGFRA; PDGFRB; PLK4; PTK2; PTK2B; PTK6; PTK7; RET; ROR1; ROR2; ROS1; RYK; SGK493; SRC; SRMS; STYK1; SYK; TEC; TEK; TEX14; TIEl; TNK1; TNK2; TNNI3K; TXK; TYK2; TYRO3; YES1; ZAP70. In a particular embodiment, the TM is a peptide, petptidomimetic, small organic molecule, antibody, aptamers, siRNA or antisense oligonucleotide.

In a particular embodiment, the tyrosine kinase is EGFR. As used herein, the term “EGFR” refers to epidermal growth factor receptor which is a member of the ErbB family of receptors, a subfamily of four closely related receptor tyrosine kinases: EGFR (ErbB-1), HER2/neu (ErbB-2), Her 3 (ErbB-3) and Her 4 (ErbB-4). EGFR are involved in the differentiation and cell growth Inhibitors of EGFR refer to compounds which inhibits cell growth. In a particular embodiment, the inhibitor of EGFR is selected from the group consisting of: gefitinib, erlotinib, afatinib, brigatinib, lapatinib, icotinib, cetuximab Osimertinib, zalutumumab, nimotuzumab, and matuzumab.

In a particular embodiment, the inhibitor of EGFR is an irreversible mutant-selective EGFR inhibitor that specifically targets EGFR-activating mutations arising de novo and upon resistance acquisition. Typically, such inhibitor inhibits the most common EGFR mutations L858R, Ex19del, and T790M. Accordingly, in a particular embodiment, the inhibitor of EGFR is EGF816 also known as Nazartinib developed by Novartis.

In a particular embodiment, the tyrosine kinase is VEGF. As used herein, the term “VEGF” refers to vascular endothelial growth factor. VEGF is involved in stimulate cellular responses by binding to tyrosine kinase receptors (the VEGFRs) on the cell surface, notably to stimulate the formation of blood vessel (angiogenesis). VEGF family comprises in mammals five members: VEGF-A, placenta growth factor (PGF), VEGF-B, VEGF-C and VEGF-D. In a particular embodiment, the inhibitors of VEGF refer to inhibit the stimulation of growth cells and formation of blood vessel. In a particular embodiment, the inhibitor of VEGF is selected from the group consisting of: ranibizumab (Lucentis®), aflibercept (Eylea®) and bevacizumab (Avastin®), Tivozanib, Lenvatinib, Axitinib, Imtinib, or brolucizumab (RTH258).

In another embodiment, the inhibitor is a VEGFR inhibitor. As used herein, the term “VEGFR” refers to receptors for vascular endothelial growth factor (VEGF). Three main subtypes of VEGFR exist: VEGFR1, VEGFR 2 and VEGFR 3. VEGFR inhibitor is selected from the group consisting of: Pegaptanib, lenvatinib, motesanib, Pazopanib, cabozantinib (Cabometyx®).

In some embodiments, the TKI is selected from the group consisting of gefitinib, erlotinib, dasatinib, nilotinib, bosutinib, ponatinib, ruxolitinib, quizartinib, cabozantinib and sunitinib. In a specific embodiment, the TKI is imatinib.

In another embodiment, the PI3K inhibitor for use according to the invention, and PARP inhibitor as a combined preparation for simultaneous, separate or sequential use in the treatment of neurofibromatosis type 1 and type 2 in a subject in need thereof.

As used herein, the term “PARP” refers to Poly (ADP-ribose) polymerase which is an enzyme involved in cellular processes such as DNA repair, genomic stability, and programmed cell death. In a particular embodiment, the PARP inhibitor is a peptide, petptidomimetic, small organic molecule, antibody, aptamers, siRNA or antisense oligonucleotide.

The PARP inhibitor is selected from the group consisting of: iniparib (BSI 201), talazoparib (also known as BMN-673), velipari (ABT-888), olaparib (also known as AZD-2281 and commercialized as Lynparza®), rucaparib (also known as Rubraca) or niraparib (also known as Zejula).

The PI3K, MAPK, PAK, mTOR, TKI, PARP and/or EGFR inhibitors as described above can be used as part of a multi-therapy for the treatment of neurofibromatosis type 1 and type 2 in a subject in need thereof.

The PI3K inhibitor can be used alone as a single inhibitor or in combination with other inhibitors like MAPK, PAK, mTOR, TKI, PARP and/or EGFR inhibitors. When several inhibitors are used, a mixture of inhibitors is obtained. In the case of multi-therapy (for example, bi-, tri- or quadritherapy), at least on other inhibitor can accompany the PI3K inhibitor.

In a particular embodiment, the PI3K and MAPK inhibitors can be combined as a bi-therapy for use in the treatment of neurofibromatosis type 1 and type 2. In a particular embodiment, the PI3K and MAPK inhibitors can be combined for use as a bi-therapy, wherein the PI3K and MAPK inhibitors are BYL719 and selumetinib respectfully.

In another embodiment, the PI3K and ERK inhibitors can be combined as a bi-therapy for use in the treatment neurofibromatosis type 1 and type 2. In a particular embodiment, the PI3K and ERK inhibitors can be combined for use as a bi-therapy, wherein the PI3K and ERK inhibitors are BYL719 and VTX-11e respectfully.

In another embodiment, the PI3K and mTOR inhibitors can be combined as a bi-therapy for use in the treatment neurofibromatosis type 1 and type 2. In a particular embodiment, the PI3K and mTOR inhibitors can be combined for use as a bi-therapy, wherein the PI3K and mTOR inhibitors are BYL719 and everolimus respectfully.

In another embodiment, the PI3K and TK inhibitors can be combined as a bi-therapy for use in the treatment neurofibromatosis type 1 and type 2. In a particular embodiment, the PI3K and TK inhibitors can be combined for use as a bi-therapy, wherein the PI3K and TK inhibitors are BYL719 and sunitinib respectfully.

In another embodiment, the PI3K and VEGF inhibitors can be combined as a as a bi-therapy for use in the treatment neurofibromatosis type 1 and type 2. In a particular embodiment, the PI3K and TK inhibitors can be combined for use as a bi-therapy, wherein the PI3K and VEGF inhibitors are BYL719 and brolucizumab (RTH258) respectfully.

In another embodiment, the PI3K, MAPK and PAK inhibitors can be combined as a tri-therapy for use in the treatment of neurofibromatosis type 1 and type 2. In a particular embodiment, the PI3K, MAPK and PAK inhibitors can be combined as a tri-therapy, wherein the PI3K, MAPK and inhibitors are BYL719, selumetinib and IPA-3 respectfully.

The present invention also relates to a method for treating neurofibromatosis type 1 and type 2 in a subject in need thereof comprising a step of administering the subject with a therapeutically effective amount of a PI3K inhibitor. In a particular embodiment, the method according to the invention, wherein the PI3K inhibitor and a MAPK inhibitor, a PAK inhibitor, an mTOR inhibitor, a TKI, a PARP inhibitor or an EGFR inhibitor, as combined preparation for use simultaneously, separately or sequentially in the treatment of neurofibromatosis type 1 and type 2.

As used herein the terms “administering” or “administration” refer to the act of injecting or otherwise physically delivering a substance as it exists outside the body (e.g., an inhibitor of PI3K alone or in a combination with at least one inhibitor selected from MAPK, PAK, mTOR, TK, and/or EGFR inhibitors) into the subject, such as by, intravenous, intramuscular, enteral, subcutaneous, parenteral, systemic, local, spinal, nasal, topical or epidermal administration (e.g., by injection or infusion). When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

A “therapeutically effective amount” is intended for a minimal amount of active agent which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder. It will be understood that the total daily usage of the compounds of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

In a second aspect, the invention relates to a pharmaceutical for use in the treatment of neurofibromatosis type 1 and type 2.

In a particular embodiment, the pharmaceutical composition according to the invention comprises a PI3K inhibitor.

In a particular embodiment, the invention relates to a pharmaceutical composition comprising a PI3K inhibitor and at least one inhibitor selected from the group consisting of: MAPK, PAK, mTOR, TK, or EGFR inhibitors as described above.

In a particular embodiment, the pharmaceutical composition according to the invention wherein the PI3K inhibitor and a MAPK inhibitor, a PAK inhibitor, an mTOR inhibitor, a TKI, or an EGFR inhibitor, as combined preparation for use simultaneously, separately or sequentially in the treatment of neurofibromatosis type 1 and type 2.

In another embodiment, the pharmaceutical composition according to the invention, wherein the PI3K inhibitor is BYL719 (Alpelisib).

The PI3K, MAPK, PAK, mTOR, TKI, PARP and EGFR inhibitors as described above may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

In some embodiments, the pharmaceutical formulation can be suitable for topical administration. In certain embodiments, the present invention provides a topical formulation comprising an inhibitor of the PI3K inhibitor and at least one inhibitor selected from the group consisting of MAPK, PAK, mTOR, TK, PARP or EGFR inhibitors. In certain embodiments, the present invention provides a topical formulation comprising a PI3K inhibitor. For example, and not by way of limitation, the present invention provides a topical formulation comprising BYL719. Dosage forms for the topical or transdermal administration of the inhibitors of the present invention include, but are not limited to, powders, sprays, ointments, pastes, creams, lotions, gels, solutions, patches and inhalants. In certain non-limiting embodiments, a topical formulation comprises one or more PI3K inhibitor comprised in micelles, liposomes, or non-lipid based microspheres. In certain non-limiting embodiments, such a topical formulation may comprise a permeability enhancing agent such as but not limited to dimethyl sulfoxide, hydrocarbons (for example, alkanes and alkenes), alcohols (for example, glycols and glycerols), acids (for example, fatty acids), amines, amides, esters (for example, isopropyl myristate), surfactants (for example, anionic, cationic, or non-ionic surfactants), terpenes, and lipids (for example, phospholipids).

In certain embodiments, the pharmaceutical formulation can be suitable for parenteral administration. The terms “parenteral administration” and “administered parenterally,” as used herein, refers to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. In certain embodiments, the present invention provides a parenteral formulation comprising a PI3K inhibitor and one of the MAPK, PAK, mTOR, TK, PARP or EGFR inhibitor as a combined preparation. In certain embodiments, the present invention provides a parenteral formulation comprising a PI3K inhibitor ans one of the MAPK, PAK, mTOR, TK, PARP or EGFR inhibitor as a combined preparation. For example, and not by way of limitation, the present invention provides a parenteral formulation comprising BYL719 and one of the MAPK, PAK, mTOR, TK, PARP or EGFR inhibitor as a combined preparation. In a particular embodiment, when the PI3K inhibitor is combined with one of the MAPK, PAK, mTOR, TK, PARP or EGFR inhibitor, the combination is formulated for oral, cutaneous or topical use.

A further object of the present invention relates to a method of screening a drug suitable for the treatment of NF1 and NF21 comprising i) providing a test compound and ii) determining the ability of said test compound to inhibit the activity of PI3K, MAPK, PAK, mTOR, TK, PARP and/or EGFR.

Any biological assay well known in the art could be suitable for determining the ability of the test compound to inhibit the activity of PI3K, MAPK, PAK, mTOR, PARP, TK, or EGFR. In some embodiments, the assay first comprises determining the ability of the test compound to bind to PI3K MAPK, PAK, mTOR, TK, or EGFR. In some embodiments, a population of cells is then contacted and activated so as to determine the ability of the test compound to inhibit the activity of PI3K, MAPK, PAK, mTOR, TK, PARP or EGFR. In particular, the effect triggered by the test compound is determined relative to that of a population of immune cells incubated in parallel in the absence of the test compound or in the presence of a control agent either of which is analogous to a negative control condition. The term “control substance”, “control agent”, or “control compound” as used herein refers a molecule that is inert or has no activity relating to an ability to modulate a biological activity or expression. It is to be understood that test compounds capable of inhibiting the activity of PI3K, as determined using in vitro methods described herein, are likely to exhibit similar modulatory capacity in applications in vivo. Typically, the test compound is selected from the group consisting of peptides, petptidomimetics, small organic molecules, aptamers or nucleic acids. For example the test compound according to the invention may be selected from a library of compounds previously synthesised, or a library of compounds for which the structure is determined in a database, or from a library of compounds that have been synthesised de novo. In some embodiments, the test compound may be selected form small organic molecules.

The invention will be further illustrated by the following figures and examples. However, these examples and figures should not be interpreted in any way as limiting the scope of the present invention.

### FIGURES

FIG. 1: NF1+/+ and NF1−/− cells were exposed to either DMSO, BYL719 at different concentrations (0.1 and 1 mmol·L−1), selumetinib at different concentrations (1 and 10 mmol·L−1) or a combination of BYL719 1 mmol·L−1 and selumetinib 10 mmol·L−1 or BYL719 1 mmol·L−1 and selumetinib 10 mmol·L−1 and IPA3. BYL719 efficiently blocks the phosphorylation of AKT (A) and S6RP (B) and induces apoptosis as assessed by PARP cleavage (C) compared to others situation. The addition of BYL719 to either selumetinib or selumetinib and IPA3 increases cells apoptosis. All data are shown as the means±s.e.m. ANOVA followed by Tukey-Kramer test. White column: NF1+/+, black column: NF1−/−.

### EXAMPLE

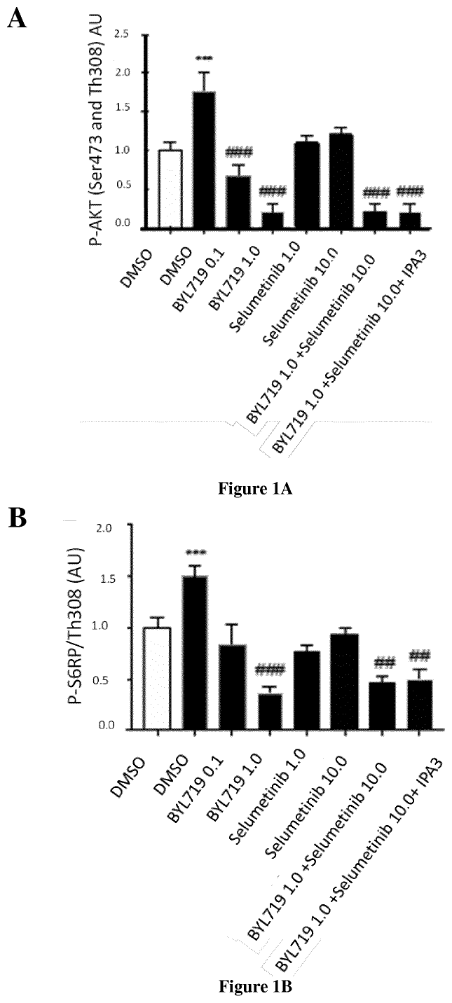

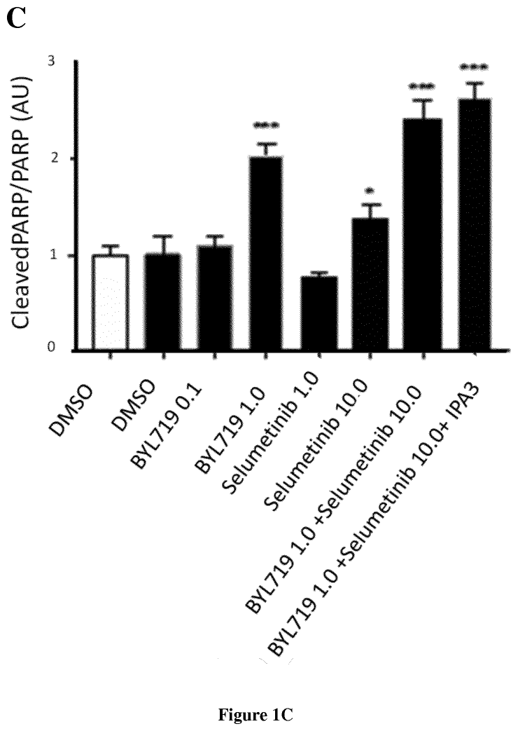

Inventors first checked in immortalized NF1−/− cells if the PIK3CA pathway was recruited (FIGS. 1A, 1B and 1C). Western blot analysis revealed that compared to WT cells, NF1−/− cells had a greater phosphorylation of AKT on both residues, Th308 and Ser473, demonstrating the spontaneous recruitment of the pathway (n=3). They then treated the cells with BYL719. After 12 h of exposure they observed that BYL719 was efficiently blocking the phosphorylation of AKT and S6RP (FIGS. 1A and 1B). PIK3CA pharmacological inhibition was associated with increased apoptosis as assessed by PARP cleavage in a dose dependent manner (FIG. 1C). They then exposed the NF1−/− cells to different dose of selumetinib, a MAP kinase inhibitor, drug that gave encouraging results in some patients with neurofibromatosis. Interestingly they observed that selumetinib induced apoptosis in a dose dependent manner but to a lower extent compared to BYL719. Based on these results, they decided to expose NF1−/− cells to a multi-targeted therapy including BYL719+selumetinib or BYL719+selumetinib+IPA-3 (PAK inhibitor). Importantly, they found that both combinations led to severe apoptosis with double strand DNA break as assessed by the phosphorylation of H2aX. Cell death in these 2 situations was more important than BYL719 or selumetinib alone.

This new approach with either BYL719 alone or in combination with other therapeutics seem to be very promising in patients with neurofibromatosis.

### REFERENCES

Throughout this application, various references describe the state of the art to which this invention pertains. The disclosures of these references are hereby incorporated by reference into the present disclosure.

1. A method for treating neurofibromatosis type 1 and type 2 in a subject in need thereof comprising administering the subject with a therapeutically effective amount of a PI3K inhibitor.

2. The method according to claim 1, further comprising administering, simultaneously, separately or sequentially, at least one inhibitor selected from the group consisting of: a MAPK inhibitor, a PAK inhibitor, an mTOR inhibitor, a TK inhibitor, a PARP inhibitor an EGFR inhibitor.

3. The method according to claim 1, wherein, the PI3K inhibitor is BYL719 (Alpelisib).

4. The method according to claim 1, wherein the PI3K inhibitor is formulated for oral, cutaneous or topical use.

5. The method according to claim 1, wherein the PI3K inhibitor is administered as part of a multi-therapy.

6. (canceled)

7. A pharmaceutical composition comprising a PI3K inhibitor and at least one inhibitor selected from the group consisting of: a MAPK inhibitor, a PAK inhibitor, an mTOR inhibitor, a TK inhibitor, a PARP inhibitor and an EGFR inhibitor.

8. (canceled)

9. The pharmaceutical composition according to claim 7, wherein the PI3K inhibitor is BYL719 (Alpelisib).

10. (canceled)
